(19) United States
(12) Patent Application Publication
(10) Pub. No.: US 2013/0280174 A1
(43) Pub. Date: Oct. 24, 2013

(54) PERSONAL CARE COMPOSITION COMPRISING METATHESIZED UNSATURATED POLYOL ESTERS

(71) Applicant: THE GILLETTE COMPANY, Boston, MA (US)

(72) Inventors: Paul Martin Lipic, West Chester, OH (US); Timothy Woodrow Coffindaffer, Maineville, OH (US); Nicole Lynette Kattau, Mason, OH (US); Karl Shiqing Wei, Mason, OH (US); Qing Stella, Cincinnati, OH (US)

(73) Assignee: The Gillette Company, Boston, MA (US)

(21) Appl. No.: 13/865,497

(22) Filed: Apr. 18, 2013

Related U.S. Application Data
(60) Provisional application No. 61/636,246, filed on Apr. 20, 2012.

(51) Int. Cl. A61K 8/37; A61Q 9/02; A61K 8/04

(52) U.S. Cl. CPC A61K 8/37; A61K 8/046; A61Q 9/02
USPC 424/43; 424/73; 424/70.15; 424/70.16; 424/70.11; 424/70.17

(57) ABSTRACT

A personal care composition which is preferably a post-foaming gel, comprising one or more oligomers derived from metathesis of unsaturated polyol esters; a water dispersible surface active agent, and a carrier comprising water.

### FIELD OF THE INVENTION

The present invention relates to a personal care composition, preferably one in the form of an aerosol shaving gel or foam which contains metathesized unsaturated polyol esters. The personal care composition is preferably a shave or hair removal preparation.

### BACKGROUND OF THE INVENTION

One of the more popular forms used today is the post foaming shave gel, developed in the late 1970's. Amine-neutralized soaps are combined with volatile hydrocarbons to form a clear, stable emulsion when kept under pressure. Once dispensed and mechanically agitated, these gels transform into thick foams.

Currently, a widely used form of shaving preparation is the type referred to as a post-foaming shave gel. These post-foaming shave gels are now well-known. See, e.g., U.S. Pat. Nos. 5,326,556 and 5,500,211. Post-foaming shave gels use a soap-based system that can optionally contain a minor amount of surfactant. Soap-based shaving gels can be extremely sensitive to even small additions of commonly used personal care benefit agents, such as, for example, polyolefins such as petrolatum and mineral oil, emollients, and vitamins. The addition of these benefit agents can cause a drastic loss of structure in the overall composition such that the end product more closely resembles a lotion than a shaving gel.

Post foaming shave gel compositions can be structured to suspend and stabilize dispersions of benefit agents while maintaining physical integrity of the compositions. The ability to deposit benefit agents and hydrate and/or condition the skin while maintaining physical integrity can be an important benefit for such compositions. Semi-solid hydrophobic components, for example, are a type of benefit agent for skin hydration and/or conditioning improvement. However, it is known that many such benefit agents can exhibit strong interactions with surfactants to cause product instability and low deposition. Petrolatum is a typical semi-solid hydrophobic ingredient capable of delivering moisturization benefits; however, when added to a post foaming shave gel, the system is not stable and the petrolatum causes the product to become runny, resulting in poor consumer acceptance of shave gels. Achieving a proper balance between stability in a composition and performance properties such as enhanced skin hydration and/or conditioning can be a difficult task, and as such, it is desirable to provide a post foaming gel composition to effectively enhance skin hydration and/or conditioning without affecting the stability of the gel causing it to be runny.

### SUMMARY OF THE INVENTION

One aspect of this invention relates to a personal care composition which is in the form of an aerosol product, preferably a post foaming gel or a shaving foam. The personal care composition comprises one or more oligomers derived from metathesis of unsaturated polyol esters in amounts by weight of composition ranging from about 0.01% to about 5%, alternatively from about 0.1% to about 1%, and alternatively from about 0.25% to about 5%. The personal care composition further comprises a water dispersible surface active agent in amounts by weight of composition ranging from about 2% to about 25%; and a carrier comprising water in amounts per weight of composition ranging from about 60% to about 93%.

### DETAILED DESCRIPTION OF THE INVENTION

While the specification concludes with the claims particularly pointing and distinctly claiming the invention, it is believed that the present invention will be better understood from the following description.

The devices, apparatuses, methods, components, and/or compositions of the present invention can include, consist essentially of, or consist of, the components of the present invention as well as other ingredients described herein. As used herein, “consisting essentially of” means that the devices, apparatuses, methods, components, and/or compositions may include additional ingredients, but only if the additional ingredients do not materially alter the basic and novel characteristics of the claimed devices, apparatuses, methods, components, and/or compositions.

All measurements used herein are in metric units unless otherwise specified.

### DEFINITIONS

As used herein, the following terms shall have the meaning specified thereafter:

The phrase “substantially free of” as used herein, unless otherwise specified means that the personal care composition comprises less than about 5%, less than about 3%, less than about 1%, or even less than about 0.1% of the stated ingredient. The term “free of” as used herein means that the personal cleansing composition comprises 0% of the stated ingredient that is the ingredient has not been added to the personal cleansing composition. However, these ingredients may incidentally form as a byproduct or a reaction product of the other components of the personal cleansing composition.

“Surfactant component” refers to a total of all anionic, nonionic, amphoteric, zwitterionic, and cationic surfactants in a phase. When calculations are based on the surfactant component, water and electrolytes can be excluded from the calculations involving the surfactant component since surfactants as manufactured can be diluted and neutralized.

The term “fatty”, as used herein, means a hydrocarbon chain having 10-22 carbon atoms (C10-22), preferably 14-18 carbon atoms (C14-18). The chain may be straight or branched and may be saturated or unsaturated (typically one or two double bonds in the chain). The term “water dispersible”, as used herein, means that a substance is either substantially dispersible or soluble in water.

The personal care composition according to the present invention may comprise from about 0.01% to about 5%, alternatively from about 0.1% to about 1%, and alternatively from about 0.25% to about 5%, of one or more oligomers derived from metathesis of unsaturated polyol esters, by weight of the personal care composition. The composition further comprises about 2% to about 25% by weight of composition, preferably about 5% to about 20% by weight of composition, of a water dispersible surface active agent, and from about 60% to about 93% by weight of composition, or from about 70% to about 85% by weight of composition of a carrier, such as water. Optionally, the composition may include lubricants, the details of which are fully described below. Preferably, the composition is in the form of a post-foaming shave gel and will additionally include about 1% to about 6% by weight of composition, preferably about 2% to about 5% by weight of composition, volatile post-foaming agent.

### 1. Metathesized Oligomer

As previously stated, the personal care composition according to the present invention may comprise one or more oligomers derived from metathesis of unsaturated polyol esters in amounts by weight of the composition ranging from about 0.01% to about 5%, alternatively from about 0.1% to about 1%, and alternatively from about 0.25% to about 5%. Exemplary metathesized unsaturated polyol esters and their starting materials are set forth in U.S. Patent Application U.S. 2009/0220443 A1, which is incorporated herein by reference.

A metathesized unsaturated polyol ester refers to the product obtained when one or more unsaturated polyol ester ingredient(s) are subjected to a metathesis reaction. Metathesis is a catalytic reaction that involves the interchange of alkylidene units among compounds containing one or more double bonds (i.e., olefinic compounds) via the formation and cleavage of the carbon-carbon double bonds. Metathesis may occur between two of the same molecules (often referred to as self-metathesis) and/or it may occur between two different molecules (often referred to as cross-metathesis). Self-metathesis may be represented schematically as shown in Equation I:

R¹—CH═CH—R²+R¹—CH═CH—R² [symbol] R¹—CH═CH—R¹+R²—CH═CH—R² (I)

where R¹ and R² are organic groups.

Cross-metathesis may be represented schematically as shown in Equation II:

R¹—CH═CH—R²+R³—CH═CH—R⁴ [symbol] R¹—CH═CH—R³+R—CH═C—R⁴+R²—CH═CH—R³R²—CH═CH—R⁴+R¹—CH═CH—R¹+R²—CH═CH—R²+R³—CH═CH—R³R⁴—CH═CH—R⁴ (II)

where R¹, R², R³, and R⁴ are organic groups.

When the unsaturated poyol ester comprises molecules that have more than one carbon-carbon double bond (i.e., a polyunsaturated polyol ester), self-metathesis results in oligomerization of the unsaturated polyol ester. The self-metathesis reaction results in the formation of metathesis dimers, metathesis trimers, and metathesis tetramers. Higher order metathesis oligomers, such as metathesis pentamers and metathesis hexamers, may also be formed by continued self-metathesis and will depend on the number and type of chains connecting the unsaturated polyol ester material as well as the number of esters and orientation of the ester relative to the unsaturation.

As a starting material, metathesized unsaturated polyol esters are prepared from one or more unsaturated polyol esters. As used herein, the term “unsaturated polyol ester” refers to a compound having two or more hydroxyl groups wherein at least one of the hydroxyl groups is in the form of an ester and wherein the ester has an organic group including at least one carbon-carbon double bond. In many embodiments, the unsaturated polyol ester can be represented by the general structure I:

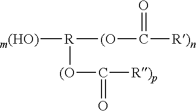

where n≧1; m≧0; p≧0; (n+m+p)≧2; R is an organic group; R′ is an organic group having at least one carbon-carbon double bond; and R″ is a saturated organic group. Exemplary embodiments of the unsaturated polyol ester are described in detail in U.S. 2009/0220443 A1.

In many embodiments of the invention, the unsaturated polyol ester is an unsaturated ester of glycerol. Sources of unsaturated polyol esters of glycerol include synthesized oils, natural oils (e.g., vegetable oils, algae oils, bacterial derived oils, and animal fats), combinations of these, and the like. Recycled used vegetable oils may also be used. Representative examples of vegetable oils include argan oil, canola oil, rapeseed oil, coconut oil, corn oil, cottonseed oil, olive oil, palm oil, peanut oil, safflower oil, sesame oil, soy-bean oil, sunflower oil, high oleoyl soy-bean oil, high oleoyl sunflower oil, linseed oil, palm kernel oil, tung oil, castor oil, high oloeyl sunflower oil, high oleoyl soybean oil, high erucic rape oils, Jatropha oil, combinations of theses, and the like. Representative examples of animal fats include lard, tallow, chicken fat, yellow grease, fish oil, combinations of these, and the like. A representative example of a synthesized oil includes tall oil, which is a byproduct of wood pulp manufacture.

Other examples of unsaturated polyol esters include diesters such as those derived from ethylene glycol or propylene glycol, esters such as those derived from pentaerythritol or dipentaerythritol, or sugar esters such as SEFOSE®. Sugar esters such as SEFOSE® include one or more types of sucrose polyesters, with up to eight ester groups that could undergo a metathesis exchange reaction. Sucrose polyesters are derived from a natural resource and therefore, the use of sucrose polyesters can result in a positive environmental impact. Sucrose polyesters are polyester materials, having multiple substitution positions around the sucrose backbone coupled with the chain length, saturation, and derivation variables of the fatty chains. Such sucrose polyesters can have an esterification (“IBAR”) of greater than about 5. In one embodiment the sucrose polyester may have an IBAR of from about 5 to about 8. In another embodiment the sucrose polyester has an IBAR of about 5-7, and in another embodiment the sucrose polyester has an IBAR of about 6. In yet another embodiment the sucrose polyester has an IBAR of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) of about 3 to about 140. In another embodiment the sucrose polyester may have an IV of about 10 to about 120. In yet another embodiment the sucrose polyester may have an IV of about 20 to 100. Further, such sucrose polyesters have a chain length of about C₁₂ to C₂₀ but are not limited to these chain lengths.

Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

Other examples of suitable natural polyol esters may include but not be limited to sorbitol esters, maltitol esters, sorbitan esters, maltodextrin derived esters, xylitol esters, and other sugar derived esters.

In other embodiments, chain lengths of esters are not restricted to C8-C22 or even chain lengths only and can include natural esters that come from co-metathesis of fats and oils with short chain olefins both natural and synthetic providing a polyol ester feedstock which can have even and odd chains as well as shorter and longer chains for the self metathesis reaction. Suitable short chain olefins include ethylene and butene.

The oligomers derived from the metathesis of unsaturated polyol esters may be further modified via hydrogenation. For example, in certain embodiments, the oligomer can be about 60% hydrogenated or more; in certain embodiments, about 70% hydrogenated or more; in certain embodiments, about 80% hydrogenated or more; in certain embodiments, about 85% hydrogenated or more; in certain embodiments, about 90% hydrogenated or more; and in certain embodiments, generally 100% hydrogenated.

In some embodiments, the triglyceride oligomer is derived from the self-metathesis of soybean oil. The soy oligomer can include hydrogenated soy polyglycerides. The soy oligomer may also include C₁₅-C₂₃ alkanes, as a byproduct. An example of metathesis derived soy oligomers is the fully hydrogenated DOW CORNING® HY-3050 soy wax, available from Dow Corning.

In other embodiments, the metathesized unsaturated polyol esters can be used as a blend with one or more non-metathesized unsaturated polyol esters. The non-metathesized unsaturated polyol esters can be fully or partially hydrogenated. Such an example is DOW CORNING® HY-3051, a blend of HY-3050 oligomer and hydrogenated soybean oil (HSBO), available from Dow Corning. In some embodiments of the invention, the non-metathesized unsaturated polyol ester is an unsaturated ester of glycerol. Sources of unsaturated polyol esters of glycerol include synthesized oils, natural oils (e.g., vegetable oils, algae oils, bacterial derived oils, and animal fats), combinations of theses, and the like. Recycled used vegetable oils may also be used. Representative examples of vegetable oils include those listed above.

Other modifications of the polyol ester oligomers can be partial amidation of some fraction of the esters with ammonia or higher organic amines such as dodecyl amine or other fatty amines This modification will alter the overall oligomer composition but can be useful in some applications providing increased lubricity of the product. Another modification can be via partial amidation of a poly amine providing potential for some pseudo cationic nature to the polyol ester oligomers. Such an example is DOW CORNING® material HY-3200. Other exemplary embodiments of amido functionalized oligomers are described in detail in WO2012006324A1, which is incorporated herein by reference.

The polyol ester oligomers may be modified further by partial hydroformylation of the unsaturated functionality to provide one or more OH groups and an increase in the oligomer hydrophilicity.

In particular embodiments, the metathesized unsaturated polyol esters and blends are formulated as small particle emulsions. An emulsion of the triglyceride oligomer can be prepared using a combination of non-ionic, zwitterionic, cationic, and anionic surfactants. In some embodiments, the emulsion of the triglyceride oligomer may be a combination of non-ionic and anionic surfactants. Suitable non-ionic emulsifiers include Neodol 1-5. Suitable anionic emulsifiers include alkyl and alkyl ether sulfates having the respective formulae ROSO₃Na and RO(C₂H₄O)ₓSO₃Na. In another embodiment, the metathesized unsaturated polyol esters are pre-melted prior to emulsification and incorporated into the personal care composition. In some embodiments of the small particle emulsions, the metathesized unsaturated polyol esters have a particle size of from about 0.05 to about 35 microns, alternatively from about 0.1 to about 10 microns, and alternatively from about 0.1 to about 2 microns.

In other embodiments, the unsaturated polyol esters and blends can be modified prior to oligomerization to incorporate near terminal branching. Exemplary polyol esters modified prior to oligomerization to incorporate terminal branching are set forth in WO2012/009525 A2, which is incorporated herein by reference.

### 2. Water Dispersible Surface Active Agent

The water dispersible surface active agent is preferably one that is capable of forming a lather and may comprise a soap, an interrupted soap, a detergent, an anionic surfactant, a non-ionic surfactant or a mixture of one or more of these. One group of suitable water dispersible surface active agent are lathering surfactants, such as those selected from the group consisting of anionic surfactants, nonionic surfactants, amphoteric surfactants, zwiterrionic surfactants, and mixtures thereof. Generally, the lathering surfactants are fairly water soluble. When used in the composition, at least about 4% of the lathering surfactants have a HLB value greater than about ten. Examples of such surfactants are found in and U.S. Pat. No. 5,624,666. Cationic surfactants can also be used as optional components, provided they do not negatively impact the overall lathering characteristics of the required lathering surfactants.

Concentrations of these surfactants are from about 1% to about 20%, alternatively from about 5% to about 25%, and alternatively from about 2% to about 30% by weight of the composition.

Suitable non-ionic surfactants will typically have an HLB of 9 or more and include the polyoxyethylene ethers of fatty alcohols, acids and amides, particularly those having 10 to 20, preferably 12 to 18, carbon atoms in the fatty moiety and about 2 to 60, preferably 4 to 30, ethylene oxide units. These include, for example, Oleth-20, Steareth-21, Ceteth-20, Glycereth-26, Laureth-4 and Laureth-23. Other non-ionic surfactants include the polyoxyethylene ethers of alkyl substituted phenols, such as Nonoxynol-4 and Nonoxynol-20, fatty alkanolamides such as Lauramide DEA and Cocamide MEA, polyethoxylated sorbitan esters of fatty acids, such as Polysorbate-20, Polysorbate-80, lauryl polyglucoside, sucrose laurate, and polyglycerol 8-oleate. Other examples of nonionic surfactants include amine oxides. Amine oxides correspond to the general formula R¹R²R³NO, wherein R¹ contains an alkyl, alkenyl or monohydroxy alkyl radical of from about 8 to about 18 carbon atoms, from 0 to about 10 ethylene oxide moieties, and from 0 to about 1 glyceryl moiety, and R² and R³ contain from about 1 to about 3 carbon atoms and from 0 to about 1 hydroxy group, e.g., methyl, ethyl, propyl, hydroxyethyl, or hydroxypropyl radicals. Examples of amine oxides suitable for use in this invention include dimethyl-dodecylamine oxide, oleyldi(2-hydroxyethyl)amine oxide, dimethyloctylamine oxide, dimethyl-decylamine oxide, dimethyl-tetradecylamine oxide, 3,6,9-trioxaheptadecyldiethylamine oxide, di(2-hydroxyethyl)-tetradecylamine oxide, 2-dodecoxyethyldimethylamine oxide, 3-dodecoxy-2-hydroxypropyldi(3-hydroxypropyl)amine oxide, dimethylhexadecylamine oxide.

Suitable amphoteric surfactants include, for example, the betaines and sultaines such as cocoamidopropyl betaine, lauramidopropyl betaine, coco dimethyl carboxymethyl betaine, coco sultaine and the like. In one embodiment, the amphoteric surfactant is a betaine selected from consisting of coco betaine, lauryl amido betaine, or a mixture thereof, and reduced salt versions thereof. Non-limiting examples of suitable zwitterionic or amphoteric surfactants are described in U.S. Pat. Nos. 5,104,646 and 5,106,609.

Anionic lathering surfactants useful in the compositions of the present invention are disclosed in McCutcheon's, Detergents and Emulsifiers, North American edition (1986), published by allured Publishing Corporation; McCutcheon's, Functional Materials, North American Edition (1992); and U.S. Pat. No. 3,929,678. Suitable anionic lathering surfactants include, for example, the sodium, potassium, ammonium and substituted ammonium salts (such as the mono-, di- and triethanolamine salts) of C8-C22, preferably C12-C18, alkyl sulfates (e.g., sodium lauryl sulfate, ammonium lauryl sulfate), alkyl sulfonates (e.g., ammonium lauryl sulfonate), alkylbenzene sulfonates (e.g. ammonium xylene sulfonate), acyl isethionates (e.g. sodium cocoyl isethionate), acyl lactylates (e.g. sodium cocoyl lactylate) and alkyl ether sulfates (e.g., ammonium laureth sulfate).

The water dispersible surface active agent can also include soaps, such as the sodium, potassium and lower alkanolamine (preferably triethanolamine) salts of C12-22, preferably C14-18, fatty acids. Typical fatty acids include lauric, myristic, palmitic and stearic acid and mixtures thereof. The preferred fatty acids are palmitic and stearic. The interrupted soaps include, for example, the sodium, potassium and lower alkanolamine (preferably triethanolamine) salts of N-fatty acyl sarcosines, wherein the fatty acyl moiety has 12 to 22, preferably 14 to 18, carbon atoms. Typical sarcosines include stearoyl sarcosine, myristoyl sarcosine, palmitoyl sarcosine, oleoyl sarcosine, lauroyl sarcosine, cocoyl sarcosine and mixtures thereof. The soaps and the interrupted soaps may be utilized in preneutralized form (i.e., as the sodium, potassium or alkanolamine salt) or in the free acid form followed by subsequent neutralization with sodium hydroxide, potassium hydroxide and/or lower alkanolamine (preferably triethanolamine) In any event, the final composition must contain sufficient base to neutralize or partially neutralize the soap component and adjust the pH to the desired level (typically between 5 and 10, more typically between 6 and 9). It is most preferred that the composition of the present invention includes a soap (e.g., triethanolamine palmitate/stearate) or an interrupted soap (e.g., triethanolamine stearoyl/myristoyl sarcosinate), or a mixture thereof.

In one embodiment, the composition is free or essentially free of soap. As used herein, “essentially free” of a component means that no amount of that component is deliberately incorporated into the composition. In one embodiment the composition is a self-foaming soap free shave gel as described in U.S. Pat. No. 5,500,211.

### 3. Lubricant

The lubricious water soluble polymer will generally have a molecular weight greater between about 300,000 and 15,000,000 daltons, preferably more than about one million daltons, and will include a sufficient number of hydrophilic moieties or substituents on the polymer chain to render the polymer water soluble. The polymer may be a homopolymer, copolymer or terpolymer. Examples of suitable lubricious water soluble polymers include polyethylene oxide, polyvinylpyrrolidone, and polyacrylamide. A preferred lubricious water soluble polymer comprises polyethylene oxide, and more particularly a polyethylene oxide with a molecular weight of about 0.5 to about 5 million daltons. Particularly suitable polyethylene oxides include, for example, PEG-14M (MW≈600,000) PEG-23M (MW≈1 million), PEG-45M (MW≈^{˜}2 million) and PEG-90M (MW≈^{˜}4 million). The lubricious water soluble polymer will generally be included in the post foaming gel composition in an amount of about 0.005% to about 3%, preferably about 0.01% to about 1%, by weight.

The water insoluble particles may include inorganic particles or organic polymer particles. Examples of inorganic particles include titanium dioxide, silicas, silicates and glass beads, with glass beads being preferred. Examples of organic polymer particles include polytetrafluoroethylene particles, polyethylene particles, polypropylene particles, polyurethane particles, polyamide particles, or mixtures of two or more of such particles. Any of the forgoing particles may also include a surface treatment to make the particles more readily dispersible or improve their cosmetic aesthetics. Preferred are polytetrafluoroethylene particles (e.g., PTFE particles available from MicroPowders, Inc. under the tradename Microslip). Preferably the water insoluble particles will have an average particle size of about 1 μm to about 100 μm, more preferably about 2 μm to about 50 μm, and most preferably about 5 μm to about 15 μm. The particles may be of any desired shape including spherical bead, elongated fiber or irregular shape, with spherical bead being the preferred shape. Generally the water insoluble particles will be included in the post foaming gel composition in an amount of about 0.01% to about 5%, preferably about 0.05% to about 2%, by weight.

The hydrogel-forming polymer is a highly hydrophilic polymer that, in water, forms organized three-dimensional domains of approximately nanometer scale. The hydrogel-forming polymer generally has a molecular weight greater than about one million daltons (although lower molecular weights are possible) and typically is at least partially or lightly crosslinked and may be at least partially water insoluble, but it also includes a sufficient number of hydrophilic moieties so as to enable the polymer to trap or bind a substantial amount of water within the polymer matrix and thereby form three-dimensional domains. It has been found that shave gel compositions that include the hydrogel-forming polymer have improved gel structure and reduced coefficient of friction (i.e., increased lubricity). Examples of suitable hydrogel-forming polymers include a polyacrylic acid or polymethacrylic acid partially esterified with a polyhydric alcohol; hydrophilic polyurethanes; lightly crosslinked polyethylene oxide; lightly crosslinked polyvinyl alcohol; lightly crosslinked polyacrylamide; hydrophobically modified hydroxyalkyl cellulose; hydroxyethyl methacrylate; and crosslinked hyaluronic acid. Generally, the hydrogel-forming polymer will be included in the post foaming gel composition in an amount of about 0.0005% to about 3%, preferably about 0.001% to about 0.5%, more preferably about 0.002% to about 0.1%, by weight.

A preferred hydrogel-forming polymer comprises polyacrylic acid partially esterified (e.g., about 40% to 60%, preferably about 50%, esterified) with glycerin. Such a polymer includes glyceryl acrylate/acrylic acid copolymer (MW>one million). It is believed that the glyceryl acrylate/acrylic acid copolymer forms a clathrate that holds water, which, upon release supplies lubrication and moisturization to the skin. A preferred source of glyceryl acrylate/acrylic acid copolymer is available from ISP Technologies, Inc. (United Guardian Inc.) under the tradename Lubrajel®, particular the form known as Lubrajel®. Most preferably, the post foaming gel composition will include about 0.25% to about 4% Lubrajel®.

The personal care composition of the present invention can also comprise a lubricant in the form of a hydrophobically modified cationic polysaccharide, modified with a hydrophobic substituent and a cationic substituent. The hydrophobically modified cationic polysaccharide is used at a level of from about 0.005% to about 3%, or from about 0.01% to about 2.0%, or from about 0.02 to about 1%, or from about 0.025% to about 0.5%, by weight. Non-limiting examples of suitable hydrophobically modified cationic polysaccharides comprise cellulose, starch and guar derivatives, particularly a derivatized hydroxyethyl cellulose ether (such as those sold under the Trade Name of SoftCAT™).

Nonlimiting examples of hydrophobically modified quaternized hydroxyethyl cellulose ethers include: those referred to in US 2007 0031362 A1 from Union Carbide, and can be referred to by those skilled in the art as SoftCAT.

Typical cellulose ethers include for example, hydroxyethyl cellulose, hydroxypropyl cellulose, methyl cellulose, hydroxypropyl methyl cellulose, hydroxyethyl methyl cellulose, hydroxyethyl carboxylmethyl cellulose, or mixtures thereof. Preferred cellulose ethers include hydroxyethyl cellulose and hydroxypropyl cellulose. Other suitable cellulose ethers comprise hydroxyethyl groups. The above cellulose ethers can be derivatized with a hydrophobic substituent and a cationic nitrogen-containing substituent to form quaternized cellulose ethers as disclosed in US Patent Published Application No. US 2011-0177018 A1, titled “Personal Care Composition Comprising A Hydrophobically Modified Cationic Polysaccharide”.

Preferred quaternized cellulose polymers with hydrophobic substitution are referred to in the industry Personal Care Products Council (formerly the Cosmetic, Toiletry, and Fragrance Association) as Polyquaternium-67 (PQ67) and are available from Dow Chemical (Amerchol Corp.) under the tradename SoftCAT™ which includes their SL, SX, and SK series polymers.

### 4. Post Foaming Agent

The post-foaming agent, when included in the post foaming gel composition, may be any volatile hydrocarbon or halohydrocarbon with a sufficiently low boiling point that it will volatilize and foam the gel upon application to the skin, but not so low that it causes the gel to foam prematurely. The typical boiling point of such an agent generally falls within the range of −20° to 40° C. Preferred post-foaming agents are selected from saturated aliphatic hydrocarbons having 4 to 6 carbon atoms, such as n-pentane, isopentane, neopentane, n-butane, isobutane, and mixtures thereof. Most preferred is a mixture of isopentane and isobutane in a weight ratio (IP:IB) of about 1:1 to about 9:1, preferably about 2:1 to about 7:1, most preferably about 3:1. The post-foaming agent will normally be selected so as to provide a vapor pressure at 20° C. of about 3 to about 20 psig, preferably about 5 to about 15 psig. The post-foaming agent will be present in an amount to provide the post foaming gel composition with a sufficiently rapid turnover—that is, transition from gel to foam when contacted with the skin—typically, in about 2 to about 30 seconds, preferably in about 5 to about 15 seconds.

### 5. Carrier

The carrier is preferably dermatologically acceptable, meaning that the carrier is suitable for topical application to the keratinous tissue, has good aesthetic properties, is compatible with the actives of the present invention and any other components, and will not cause any safety or toxicity concerns. In one embodiment, the post foaming gel composition comprises from about 50% to about 99.99%, preferably from about 60% to about 93%, more preferably from about 70% to about 90%, and even more preferably from about 80% to about 85% of the carrier by weight of the composition. In one embodiment, the carrier comprises water.

### 6. Other Adjunct Ingredients

Although not necessary to forming a useful shave gel composition, other cosmetic ingredients may be advantageously added to improve the application aesthetics and/or achieve other shave benefits. For example, the composition may include one or more of the following components: beard wetting agents, skin conditioning agents (e.g., vitamins A, C and E, aloe, allantoin, panthenol, alpha-hydroxy acids, phospholipids, triglycerides, botanical oils, amino acids), foam boosters, emollients, humectants (e.g., glycerin, sorbitol, propylene glycol), fragrances, colorants, antioxidants, preservatives, etc. It is particularly preferred to include glycerin in the shave gel composition of the present invention, preferably in an amount of about 0.1% to about 10%, more preferably about 0.3% to about 1%, by weight. Glycerin improves the emolliency of the composition.

It may be advantageous to include a sorbitan fatty ester or a sucrose fatty ester, typically in an amount of about 0.1% to about 3%, preferably about 0.3% to about 2%, by weight. These materials have multifunctional properties of emulsifier, moisturizer and anti-irritant. Sorbitan fatty esters include sorbitan stearate, sorbitan oleate, sorbitan isostearate, sorbitan laurate, sorbitan dioleate, etc. Sucrose fatty esters include sucrose stearate, sucrose oleate, sucrose isostearate, sucrose cocoate, sucrose distearate, etc. The sorbitan esters and sucrose esters may be mixtures of mono-, di- and tri-esters.

It may also be desirable to include an ester of a fatty acid, typically in an amount of about 0.5% to about 5%, preferably about 1% to about 4%, by weight. Useful fatty esters include glyceryl fatty esters such as, for example, glyceryl oleate and glyceryl dioleate, and fatty alcohol esters such as, for example, isostearyl linoleate, isocetyl oleate, and isostearyl isostearate. These materials provide emolliency, lubrication and gel structure.

It may also be desirable to include one or more of the sensates or excipients suitable for use on skin. These sensates or excipients can be those which are commonly used in cosmetic and personal care compositions on the market today. Each of the additives can be provided at from about 0.001% to about 10%, or from about 0.1% to about 5% by weight of the composition. Non-limiting examples of suitable additives include one or more of: Bisabolol and Ginger root; sodium polyethylene glycol 7 olive oil carboxylate; Lauryl p-Cresol Ketoxime, 4-(1-Phenylethyl)1,3-benzenediol, Lupin (Lupinus albus) oil & wheat (Triticum vulgare) germ oil unsaponifiables, Hydrolyzed lupin protein, Extract of L-lysine and L-arginine peptides, Oil soluble vitamin C, Evodia rutaecarpa fruit extract, Zinc pidolate and zinc PCA, Alpha-linoleic acid, p-thymol, extract of camellia sinensis (such as white tea extract); panthenol; glycyrrhizinate salts, and combinations thereof; and skin and/or hair care active selected from the group consisting of sugar amines, vitamin B₃, retinoids, hydroquinone, peptides, farnesol, phytosterol, dialkanoyl hydroxyproline, hexamidine, salicylic acid, N-acyl amino acid compounds, sunscreen actives, water soluble vitamins, oil soluble vitamins, hesperedin, mustard seed extract, glycyrrhizic acid, glycyrrhetinic acid, carnosine, Butylated Hydroxytoluene (BHT) and Butylated Hydroxyanisole (BHA), menthyl anthranilate, cetyl pyridinium chloride, tetrahydrocurmin, vanillin or its derivatives, ergothioneine, melanostatine, sterol esters, idebenone, dehydroacetic acid, Licohalcone A, creatine, creatinine, feverfew extract, yeast extract (e.g., Pitera®), beta glucans, alpha glucans, diethylhexyl syringylidene malonate, erythritol, p-cymen-7-ol, benzyl phenylacetate, 4-(4-methoxyphenyl)butan-2-one, ethoxyquin, tannic acid, gallic acid, octadecenedioic acid, p-cymen-5-ol, methyl sulfonyl methane, an avenathramide compound, fatty acids (especially poly-unsaturated fatty acids), anti-fungal agents, thiol compounds (e.g., N-acetyl cysteine, glutathione, thioglycolate), other vitamins (vitamin B 12), beta-carotene, ubiquinone, amino acids, their salts, their derivatives, their precursors, and/or combinations thereof; and a dermatologically acceptable carrier. These and other potentially suitable actives are described in greater detail in U.S. Patent Publication No. 2008/0069784 and U.S. Ser. No. 61/364,932 and U.S. Ser. No. 12/984,958.

In another embodiment, the personal care composition further comprises a sensate. A non-limiting example of a suitable sensates is methyl naphthalenyl ketone. In one embodiment the composition comprises from about 0.001% to about 1% of methyl naphthalenyl ketone. The methyl naphthalenyl ketone can be a 1-(1,2,3,4,5,6,7,8-octahydro-2,3,8,8-tetramethyl-2naphthalenyl)-ethan-1-one molecule or an isomer or derivative thereof. Commercially available as Iso-E-Super from IFF of New York.

In yet another embodiment, the personal care composition further comprising from about 0.001% to about 1%, preferably from about 0.05% to about 0.5% of a cooling agent. Preferred cooling agents but not limited to are menthol, CoolAct 10, menthyl lactate, menthone glycerin acetal and combinations thereof. It may also be desirable to include one or more silicone polymers comprising any member of the dimethicone or dimethiconol family. Non-limiting examples of suitable additions include one or more of: dimethicone, dimethiconol, trimethylsiloxane, polydimethylsiloxane, silicone elastomer and combinations thereof. Each of the additives can be provided at from about 0.01% to about 10%, preferably about 0.5% to about 5% by weight.

It may also be desirable to include one or more hollow particles products such as Expancel Wetted 920-WE40_D24 commercially marketed, typically in an amount from 0.01% to 10%, preferably about 1% to about 5% by weight.

It may further be desirable to include a propoxylated fatty amide, typically in an amount of about 0.5% to about 5%, preferably about 1% to about 3%, by weight. The propoxylated fatty amide will typically have from 1 to 3 propoxyl groups attached to a hydroxyloweralkyl fatty amide. Thus, suitable propoxylated fatty amides include, for example, PPG-2-hydroxyethyl coco/isostearamide, PPG-3-hydroxyethyl linoleamide, and PPG-2-hydroxyethyl cocamide.

The compositions of the present invention can comprise one or more thickening agents, preferably from about 0.05% to about 10%, more preferably from about 0.1% to about 5%, and even more preferably from about 0.25% to about 4%, by weight of the composition. Nonlimiting classes of thickening agents include those selected from the group consisting of: Carboxylic Acid Polymers (crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol); Crosslinked Polyacrylate Polymers (including both cationic and nonionic polymers, such as described in U.S. Pat. Nos. 5,100,660; 4,849,484; 4,835,206; 4,628,078; 4,599,379, and EP 228,868); Polymeric sulfonic acid (such as copolymers of acryloyldimethyltaurate and vinylpyrrolidone) and hydrophobically modified polymeric sulfonic acid (such as crosspolymers of acryloyldimethyltaurate and beheneth-25 methacrylate); Polyacrylamide Polymers (such as nonionic polyacrylamide polymers including substituted branched or unbranched polymers such as polyacrylamide and isoparaffin and laureth-7 and multi-block copolymers of acrylamides and substituted acrylamides with acrylic acids and substituted acrylic acids); Polysaccharides (nonlimiting examples of polysaccharide gelling agents include those selected from the group consisting of cellulose, carboxymethyl hydroxyethylcellulose (sold under the trademarks “Natrosol”), cellulose acetate propionate carboxylate, hydroxyethylcellulose, hydroxyethyl ethylcellulose, hydroxypropylcellulose (sold under the trademarks “Klucel”), hydroxypropyl methylcellulose, methyl hydroxyethylcellulose, microcrystalline cellulose, sodium cellulose sulfate, and mixtures thereof); Gums (i.e. gum agents such as acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboyxmethyl dextran, sodium carrageenan, tragacanth gum, xanthan gum, and mixtures thereof); and crystalline, hydroxyl-containing fatty acids, fatty esters or fatty waxes (such as microfibrous bacterial cellulose structurants as disclosed in U.S. Pat. Nos. 6,967,027 to Heux et al.; 5,207,826 to Westland et al.; 4,487,634 to Turbak et al.; 4,373,702 to Turbak et al. and 4,863,565 to Johnson et al., U.S. Patent Publ. No. 2007/0027108 to Yang et al.)

The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, fatty alcohols and fatty acids, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof. Additional non-limiting examples of additional suitable skin treatment actives are included in U.S. 2003/0082219 in Section I (i.e. hexamidine, zinc oxide, and niacinamide); U.S. Pat. No. 5,665,339 at Section D (i.e. coolants, skin conditioning agents, sunscreens and pigments, and medicaments); and US 2005/0019356 (i.e. desquamation actives, anti-acne actives, chelators, flavonoids, and antimicrobial and antifungal actives). Examples of suitable emulsifiers and surfactants can be found in, for example, U.S. Pat. No. 3,755,560, U.S. Pat. No. 4,421,769, and McCutcheon's Detergents and Emulsifiers, North American Edition, pages 317-324 (1986). It should be noted, however, that many materials may provide more than one benefit, or operate via more than one mode of action. Therefore, classifications herein are made for the sake of convenience and are not intended to limit the active to that particular application or applications listed. Other useful optional ingredients include: Anti-Wrinkle Actives and/or Anti-Atrophy Actives; Anti-Oxidants and/or Radical Scavengers; Anti-Inflammatory Agents; Anti-Cellulite Agents; Tanning Actives; Skin Lightening Agents; Sunscreen Actives; Water Soluble Vitamins; particulates; and combinations thereof.

The composition can also include other commonly included ingredients which are used in commercially available post foaming shave gels such as those described in US Patent Publ. Nos. 2006/0257349, 2006/0257350, 2005/0175575 and 2011/0274627.

### 7. Product Forms and Uses

The personal care compositions of the present invention can be used for as a hair removal preparation such as a post foaming shave gel. The present composition may be formulated as an aerosol foam, a post-foaming gel (which is the preferred form) or a non-aerosol gel or lather. It may be packaged in any suitable dispenser normally used for dispensing personal care compositions (such as shaving compositions). These include collapsible tubes, pump or squeeze containers, and aerosol-type dispensers, particularly those with a barrier to separate the post foaming gel composition from the propellant required for expulsion.

The latter type of dispensers include: (1) mechanically pressurized bag-in-sleeve systems in which a thin-walled inner bag containing the product is surrounded by an outer elastic sleeve that is expanded during the product filling process and provides dispensing power to expel the product (e.g., the ATMOS System available commercially from the Exxel Container Co.); (2) (a) a container preform comprising a polymeric preform and an elastically deformable band surrounding at least a portion of the polymeric perform such as described in U.S. 2009/0263174 to Chan et al; (3) manually activated air pump spray devices in which a pump system is integrated into the container to allow the user to pressurize the container with air in order to expel the product (e.g., the “AIRSPRAY” system available from Airspray International); (4) piston barrier systems in which the product is separated from the driving means by a tight-fitting piston which seals to the side of the container and may be driven by a spring under tension, by a vacuum on the product side of the piston, by finger pressure, by gas pressure to the piston, or by a variety of other means known to the packaging industry; and (5) bag-incan (SEPRO) systems in which the product is contained in a flexible bag within a can, with a suitable propellant injected into the space between the can and the flexible bag. It is preferred to protect the composition from oxidation and heavy metal contamination. This can be achieved, for example, by purging the composition and container with nitrogen to remove oxygen and by utilizing inert containers (e.g., plastic bottles or bags, aluminum cans or polymer coated or lined cans).

The present composition can be used in combination with various hair removal applications (prior to, concurrently with, and/or after), including but not limited to shaving (wet or dry shaving, via electric razors, via powered or manual razors which can be reuseable or disposable, and combinations thereof), epilation, electrolysis, wax or depilatories as well as energy delivery devices to help regulate hair growth. Nonlimiting examples of energy deliver devices include: light, heat, sound (including ultrasonic waves and radio frequency), electrical energy, magnetic energy, electromagnetic energy (including radiofrequency waves and microwaves), and combinations thereof. The light energy may be delivered by devices including, but not limited to, lasers, diode lasers, diode laser bars, diode laser arrays, flash lamps, intense pulsed light (IPL) sources, and combinations thereof. See e.g. US2006/0235370A1.

### 8. Gel Stiffness

Gel stiffness is a material property relevant to the consumer acceptance of a product. Typical well accepted gel stiffness values are >50 g and <250 g. A product with a gel stiffness <30 g may be described as ‘runny’ Preferably the gel stiffness ranges from about 60 g to about 180 g. Gel stiffness is measured using a Texture Analyzer model TA-1000 equipment, fitted with an end probe that is 1″ in diameter and ¼″ thick and constructed of aluminum. Samples to be tested are first equilibrated at 21° C. for 2 hours, then dispensed in to a sampling container with a 1.50″ inner diameter and a depth of 0.945″. The top surface of the sample is scraped even with the sampling container.

Center the sampling container directly under the probe, ½″-1″ from the probe. Within 30 seconds start the experiment. Probe speed: 2 mm/s, probe stroke: 19 mm after contact with top surface. Record the instrument reading of force (g) at the end of the stroke—this is the gel stiffness.

### 9. Methods of Making

Those of ordinary skill in the art will understand that the personal care composition can be made in a variety of known ways for making similar compositions. In one embodiment, where the personal care composition is a aerosol, such as a post foaming shave gel, the method of making is as described in U.S. Pat. No. 7,820,152.

The water soluble polymers (such as the hydrophobically modified cationic polysaccharide of the present invention, polyethylene oxide, hydroxyethylcellulose) and sorbitol are added to water and mixed until the polymers are completely dissolved (about 30 min.). The aqueous mixture is then heated and the glyceryl oleate and fatty acids are added at about 60° C. and well mixed while the heating continues. At 80-85° C. the triethanolamine is added and mixed for about 20 minutes to form the aqueous soap phase. The semi-solid hydrophobic ingredient is added when the phase still at elevated temperatures (>70° C.) to enable it to melt and blend in to the soap phase. After cooling the aqueous soap phase to room temperature, the remaining components (i.e., Lubrajel Oil, glycerin, fragrance, colorant, botanicals) are added to the aqueous soap phase and mixed well to form the gel concentrate. (Water may be added if required to bring the batch weight to 100%, thereby compensating for any water loss due to evaporation.) The concentrate is then combined with the volatile post-foaming agent under pressure within the filling line and filled into Bag-on-Valve aerosol cans with shearing through the valve under nitrogen pressure.

### 10. Comparative Examples

The following examples in Table 1 is formulated as described below. Making instructions for Examples 1-7 can be found in the second paragraph of section 9 of this application. QS means quantity sufficient to reach 100%. All values are percent by weight.

TABLE 1

| Example | 1 | 2 | 3 | 4 | 5 | 6 |
|---|---|---|---|---|---|---|
| Palmitic acid | 7.5291% | 7.5291% | 7.5291% | 7.5291% | 7.5291% | 7.5291% |
| Stearic Acid | 2.5259% | 2.5259% | 2.5259% | 2.5259% | 2.5259% | 2.5259% |
| Glyceryl Oleate | 1.9430% | 1.9430% | 1.9430% | 1.9430% | 1.9430% | 1.9430% |
| Triethanolamine (99%) | 6.1690% | 6.1690% | 6.1690% | 6.1690% | 6.1690% | 6.1690% |
| Sorbitol 70% Solution | 0.9715% | 0.9715% | 0.9715% | 0.9715% | 0.9715% | 0.9715% |
| Glycerin | 0.4857% | 0.4857% | 0.4857% | 0.4857% | 0.4857% | 0.4857% |
| hydroxyethyl cellulose¹ | 0.4857% | 0.4857% | 0.4857% | 0.4857% | 0.4857% | 0.4857% |
| PEG-90M² | 0.0874% | 0.0874% | 0.0874% | 0.0874% | 0.0874% | 0.0874% |
| Fragrance | 0.9715% | 0.9715% | 0.9715% | 0.9715% | 0.9715% | 0.9715% |
| Hydrogenated soy bean oil | 0.4858% | 4.8575% | 9.7150% | | | |
| and soy oligomer blend³ | | | | | | |
| Petrolatum⁴ | | | | 0.4858% | 4.8575% | 9.7150% |
| Isopentane (and) Isobutane | 2.8500% | 2.8500% | 2.8500% | 2.8500% | 2.8500% | 2.8500% |
| Water | q.s to | q.s to | q.s to | q.s to | q.s to | q.s to |
| | 100% | 100% | 100% | 100% | 100% | 100% |

Physical Properties

| | | | | | | |
|---|---|---|---|---|---|---|
| Gel strength (g) | 86 | 47 | 50 | 25 | 14 | 14 |

¹Available as Natrosol 250 HHR from Hercules Inc., Wilmington, DE
²Available as Polyox WSR-301 from Amerchol Corp., Piscataway, NJ
³Available as HY-3051 Soy Wax Blend from Dow Corning Corp., Midland, MI
⁴Available as Protopet from Sonneborn Inc., Mahwah, NJ

### Additional Examples

| Example | 7 | 8 | 9 | 10 | 11 |
|---|---|---|---|---|---|
| Sorbitol 70% Solution | 0.9600% | 0.9615% | 0.9715% | 0.9700% | 0.9715% |
| Glycerin | 4.8000% | 4.8075% | 0.4857% | 0.4850% | 0.4857% |
| hydroxyethyl cellulose¹ | 0.4800% | 0.3846% | 0.4857% | 0.7275% | 0.4857% |
| PEG-90M² | 0.1632% | 0.0577% | 0.1652% | 0.1067% | 0.1652% |
| PEG-23M³ | 0.0480% | 0.0865% | 0.0486% | 0.0582% | 0.0486% |
| PTFE⁴ | 0.1440% | 0.0481% | 0.1457% | 0.1940% | 0.1457% |
| Palmitic acid | 7.4400% | 7.4516% | 7.5291% | 6.3923% | 7.5291% |
| Stearic Acid | 2.4960% | 2.4999% | 2.5259% | 2.1437% | 2.5259% |
| Glyceryl Oleate | 1.3920% | 2.8845% | 1.9430% | 2.4250% | 1.9430% |
| Triethanolamine (99%) | 6.0960% | 6.1055% | 5.8776% | 5.2380% | 6.1690% |
| Lubrajel Oil⁵ | 0.9600% | 0.7211% | 0.9715% | 1.2125% | 0.9715% |
| Fragrance | 1.2960% | 0.7692% | 1.0687% | 0.9700% | 1.3115% |
| Dye | 0.0029% | 0.0025% | 0.0008% | | |
| Menthol | | 0.0481% | | 0.0970% | 0.2429% |
| Hydrogenated soy bean oil and | 7.2000% | 3.8460% | 9.7150% | 9.7000% | 6.8005% |
| soy oligomer blend⁶ | | | | | |
| Expancel⁷ | | | | 1.9400% | |
| Dimethicone⁸ | 1.9200% | 3.8460% | 1.9430% | | 2.9145% |
| Iso E Super⁹ | 0.0576% | 0.1923% | 0.0583% | 0.0582% | 0.1457% |
| PPG-15 Stearyl Ether¹⁰ | 0.2400% | 0.3365% | | 0.2425% | 0.3400% |
| Isopentane (and) Isobutane | 4.0000% | 0.9615% | 2.8500% | 3.0000% | 2.8500% |
| Water | q.s to 100% | q.s to 100% | q.s to 100% | q.s to 100% | q.s to 100% |
| Example | 12 | 13 | 14 | 15 | 16 |
| Sorbitol 70% Solution | 0.9600% | 0.9600% | 0.9725% | 0.9625% | 0.9715% |
| Glycerin | 0.4800% | 0.4800% | 0.4863% | 9.6250% | 0.4857% |
| hydroxyethyl cellulose¹ | 0.4800% | 0.4800% | 0.4863% | 0.4813% | 0.4857% |
| PEG-90M² | 0.1632% | 0.1632% | 0.1653% | 0.1636% | 0.1652% |
| PEG-23M³ | 0.0480% | 0.0480% | 0.0584% | 0.0578% | |
| PTFE⁴ | 0.1440% | 0.1440% | 0.1459% | 0.1444% | |
| Palmitic acid | 7.4400% | 7.4400% | 9.0443% | 7.4594% | 7.5291% |
| Stearic Acid | 2.4960% | 2.4960% | 3.0342% | 2.5025% | 2.5259% |
| Glyceryl Oleate | 1.7280% | 1.7280% | 0.9725% | 1.7325% | 1.9430% |
| Triethanolamine (99%) | 6.0960% | 6.0960% | 7.4105% | 6.1119% | 6.1690% |
| Lubrajel Oil⁵ | 0.9600% | 0.9600% | | 0.9625% | |
| Fragrance | 1.2960% | 1.2960% | 0.5835% | 1.2994% | 0.7692% |
| Dye | 0.0008% | | 0.0022% | | 0.0022% |
| Menthol | 0.2208% | 0.2208% | | | 0.2429% |
| Hydrogenated soy bean oil and | 4.8000% | 4.8000% | 7.7800% | 4.8125% | 7.7800% |
| soy oligomer blend⁶ | | | | | |
| Expancel⁷ | 0.9600% | 0.9600% | | 0.9625% | |
| Dimethicone⁸ | 2.8800% | 2.8800% | | 1.9250% | 2.9145% |
| Iso E Super⁹ | 0.1440% | 0.1440% | | 0.1925% | |
| PPG-15 Stearyl Ether¹⁰ | 0.3360% | 0.3360% | | 0.3369% | 0.3400% |
| Isopentane (and) Isobutane | 4.0000% | 4.0000% | 2.7500% | 3.7500% | 2.8500% |
| Water | q.s to 100% | q.s to 100% | q.s to 100% | q.s to 100% | q.s to 100% |

¹Available as Natrosol 250 HHR from Hercules Inc., Wilmington, DE
²Available as Polyox WSR-301 from Amerchol Corp., Piscataway, NJ
³Available as Polyox WSR N-12K from Amerchol Corp., Piscataway, NJ
⁴Available as Microslip 519 from Micro Powders Inc., Tarrytown, NY
⁵Available from Guardian Laboratories, Hauppauge, NY
⁶Available as HY-3051 Soy Wax Blend from Dow Corning Corp., Midland, MI
⁷Available from AkzoNobel., Bridgewter, NJ
⁸Available as Xiameter(R) PMX-200 Silicone Fluid from Dow Corning Corp., Midland, MI
⁹Available from International Flavors & Fragrances Inc., Shrewsbury, NJ
¹⁰Available as Arlamol PS15E from Croda, Inc., Edison, NJ

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm” All measurements are performed at 23° C., unless otherwise specified.

All documents cited in the DETAILED DESCRIPTION OF THE INVENTION are, in the relevant part, incorporated herein by reference; the citation of any document is not to be construed as an admission that it is prior art with respect to the present invention. To the extent that any meaning or definition of a term or in this written document conflicts with any meaning or definition in a document incorporated by reference, the meaning or definition assigned to the term in this written document shall govern.

Except as otherwise noted, the articles “a,” “an,” and “the” mean “one or more.”

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A personal care composition comprising:
a. from about 0.01% to about 5% by weight of the personal care composition, one or more oligomers derived from metathesis of unsaturated polyol esters;
b. from about 2% to about 25% by weight of the personal care composition, a water dispersible surface active agent; and
c. from about 60% to about 93% by weight of the personal care composition, a carrier comprising water.

2. The personal care composition of claim 1, further comprising from about 1% to about 6% of a volatile post-foaming agent.

3. The personal care composition of claim 1 wherein the personal care composition is a post foaming shave gel or an aerosol foam.

4. The personal care composition of claim 1, wherein the one or more oligomers is a triglyceride oligomer.

5. The personal care composition of claim 4, wherein the triglyceride oligomer is a soy oligomer.

6. The personal care composition of claim 5, wherein the soy oligomer is a fully hydrogenated soy oligomer.

7. The personal care composition of claim 5, wherein the soy oligomer is about 80% hydrogenated or more.

8. The personal care composition of claim 1 comprising from about 0.1% to about 1% by weight of the personal care composition, one or more oligomers derived from metathesis of unsaturated polyol esters.

9. The personal care composition of claim 1 wherein the one or more oligomers comprises hydrogenated soy polyglycerides.

10. The personal care composition of claim 1 wherein the one or more oligomers derived from metathesis of unsaturated polyol esters comprises a blend with one or more non-metathesized unsaturated, partially hydrogenated or fully hydrogenated polyol ester.

11. The personal care composition of claim 10 wherein the one or more oligomers derived from metathesis of unsaturated polyol esters are soy based and the non-metathesized polyol ester is soy based.

12. The personal care composition of claim 1, further comprising a lubricant, the lubricant comprises at least one of: a lubricious water soluble polymer, a water insoluble particle, a hydrogel-forming polymer, and a mixture thereof.

13. The personal care composition of claim 12, wherein the lubricant comprises at least one of: from about 0.01% to about 1%, of a lubricious water soluble polymer, from about 0.01% to about 5% of a water insoluble particle, from about 0.0005% to about 3%, of a hydrogel-forming polymer, and a mixture thereof, by weight.

14. The personal care composition of claim 12, wherein the lubricious water soluble polymers comprises at least one of a polyethylene oxide, a polyvinylpyrrolidone, a polyacrylamide, and a mixture thereof.

15. The personal care composition of claim 12, wherein the water insoluble particles comprises at least one of inorganic particles, organic polymer particles, and a mixture thereof.

16. The personal care composition of claim 12, wherein the hydrogel-forming polymers comprises at least one of: a polyacrylic acid or polymethacrylic acid partially esterified with a polyhydric alcohol; a hydrophilic polyurethanes; a lightly crosslinked polyethylene oxide; a lightly crosslinked polyvinyl alcohol; a lightly crosslinked polyacrylamide; a hydrophobically modified hydroxyalkyl cellulose; a hydroxyethyl methacrylate; and crosslinked hyaluronic acid.

17. The personal care composition of claim 1, wherein the water dispersable surface active agent comprises a lathering surfactant other than a non-ionic surfactant.

18. The personal care composition of claim 1, wherein the water dispersible surface active agent is free or essentially free of a soap.

19. The personal care composition of claim 1 wherein the one or more oligomers are self metathesized.
